(19) United States
(12) Patent Application Publication
(10) Pub. No.: US 2022/0175527 A1
(43) Pub. Date: Jun. 9, 2022

(54) DELIVERY SYSTEMS FOR PROSTHETIC HEART VALVES

(71) Applicant: Medtronic, Inc., Minneapolis, MN (US)

(72) Inventors: Constantin Ciobanu, Lackagh (IE); Thomas Maxwell, Boston, MA (US); Evelyn Birmingham, Bothermore (IE); Niall Crosbie, Clare (IE); Emma Keane, Galway (IE); Francis Glynn, Loughrea (IE); Shawnee Kathleen Black, Coleraine (IE); Tomas Gilson, Galway (IE); Patrick King, Gort (IE); Stephen Alexander Montgomery, Galway (IE); Ricky Unadkat, Galway City (IE); Nicholas Fox, Oranmore (IE); Jake Dunlea, Shannon (IE); Timothy Desmond Farrell, Tralee (IE); Austin Berg, Cambridge, MA (US)

(21) Appl. No.: 17/543,230

(22) Filed: Dec. 6, 2021

Related U.S. Application Data
(60) Provisional application No. 63/122,440, filed on Dec. 7, 2020.

(51) Int. Cl. A61F 2/24; A61M 25/01; A61M 25/00; A61M 39/22

(52) U.S. Cl. CPC A61F 2/2436; A61M 25/0136; A61M 25/0045; A61M 25/0053; A61M 2039/229; A61M 39/223; A61M 25/0097; A61M 25/0009; A61M 2025/0004; A61M 25/0029

(57) ABSTRACT

A system for delivering an implantable medical device to an implant location includes a control handle portion, a catheter portion coupled to the control handle portion, and a distal portion configured to receive the implantable medical device. The catheter portion includes outer shaft including an inner braided layer extending axially along the outer shaft, an outer braided layer extending axially along the outer shaft, wherein the outer braided layer has a lower density of braids relative to the inner braided layer, and an axial spine positioned between the inner braided layer and the outer braided layer extending axially along the outer shaft.

### CROSS-REFERENCE TO RELATED APPLICATIONS

This application claims the benefit under 35 U.S.C. § 119(e) of the filing date of U.S. Provisional Application No. 63/122,440, filed Dec. 7, 2020, the contents of which are incorporated by reference herein in their entirety.

### FIELD

The present technology is generally related to medical devices. And, more particularly, to delivery systems and methods for stents, prosthetic heart valves and other implantable medical devices.

### BACKGROUND

Patients suffering from various medical conditions or diseases may require surgery to install an implantable medical device. For example, valve regurgitation or stenotic calcification of leaflets of a heart valve may be treated with a heart valve replacement procedure. A traditional surgical valve replacement procedure requires a sternotomy and a cardiopulmonary bypass, which creates significant patient trauma and discomfort. Traditional surgical valve procedures may also require extensive recuperation times and may result in life-threatening complications.

One alternative to a traditional surgical valve replacement procedure is delivering implantable medical devices using minimally-invasive techniques. For example, a prosthetic heart valve can be percutaneously and transluminally delivered to an implant location. In such methods, the prosthetic heart valve can be compressed or crimped on a delivery catheter for insertion within a patient's vasculature; advanced to the implant location; and re-expanded to be deployed at the implant location. Among devices commonly used to access vascular and other locations within a body and to perform various functions at those locations are medical catheters, or delivery catheters, adapted to deliver and deploy medical devices such as prosthetic heart valves, stent-grafts, and stents to selected targeted sites in the body. Such medical devices typically are releasably carried within a distal region of the delivery catheter in a radially compressed delivery state or configuration as the catheter is navigated to and positioned at a target treatment/deployment site. In many cases, such as those involving cardiovascular vessels, the route to the treatment/deployment site may be tortuous and may present conflicting design considerations requiring compromises between dimensions, flexibilities, material selection, operational controls and the like.

Typically, advancement of a delivery catheter within a patient is monitored fluoroscopically to enable a clinician to manipulate the catheter to steer and guide its distal end through the patient's vasculature to the target treatment/deployment site. This tracking requires a distal end of the delivery catheter to be able to navigate safely to the target treatment/deployment site through manipulation of a proximal end by the clinician. Such manipulation may encompass pushing, retraction and torque forces or a combination of all three. It is therefore required for the distal end of the delivery catheter to be able to withstand all these forces.

A delivery catheter desirably will have a low profile/small outer diameter to facilitate navigation through tortuous vasculature; however, small outer diameter catheters present various design difficulties resulting from competing considerations, resulting in design trade-offs. For instance, such delivery catheters must be flexible enough to navigate the tortuous vasculature or anatomy of a patient. However, typical constructions of delivery catheters must attempt to balance a requisite flexibility, with axial strength/stiffness (the property that permits the delivery catheter to be pushed and pulled) and torsional strength/stiffness (the property that permits the delivery catheter to be rotated about its longitudinal axis). It is especially important to balance these properties in a distal portion of the delivery catheter within which a prosthesis is held in its radially compressed, delivery state.

A need in the art still generally exists for improved catheters configured to navigate through or within a patient's anatomy.

### SUMMARY

The techniques of this disclosure generally relate to delivery systems of implantable medical devices.

In an aspect of the present disclosure, a system for delivering an implantable medical device to an implant location includes a control handle portion, a catheter portion coupled to the control handle portion, and a distal portion for receiving the implantable medical device. The catheter portion includes an outer shaft including an inner braided layer extending axially along the outer shaft, an outer braided layer extending axially along the outer shaft, wherein the outer braided layer has a lower density of braids relative to the inner braided layer, and an axial spine positioned between the inner braided layer and the outer braided layer extending axially along the outer shaft

In another aspect hereof, in the system in accordance with any other aspect hereof, the axial spine comprises a single wire axially extending the length of the outer shaft.

In another aspect hereof, in the system in accordance with any other aspect hereof, the outer shaft includes a proximal portion including a first jacket layer surrounding the inner braided layer, the outer braided layer and the axial spine, a distal portion positioned distal to the proximal portion and including a second jacket layer surrounding the inner braided layer, the outer braided layer and the axial spine, and a middle portion positioned between the proximal portion and the distal portion and including a third jacket layer surrounding the inner braided layer, the outer braided layer and the axial spine surrounding the inner braided layer, the outer braided layer and the axial spine.

In another aspect hereof, in the system in accordance with any other aspect hereof, the first jacket layer, the second jacket layer, the third jacket layer comprise different materials.

In another aspect hereof, in the system in accordance with any other aspect hereof the catheter portion further includes a stability shaft coupled to the control handle portion and surrounding the outer shaft, wherein the stability shaft extends from the control handle portion to a distal position on the outer shaft.

In another aspect hereof, in the system in accordance with any other aspect hereof the distal position is approximately 36 inches from the control handle portion.

In another aspect hereof, the system in accordance with any other aspect hereof further includes a capsule coupled to a distal end of the outer shaft, wherein the capsule is actuatable to move axially from the distal portion to expose the implantable medical device.

In another aspect hereof, in the system in accordance with any other aspect hereof, the capsule includes a flexible region, the flexible region having a stiffness that is less than other regions of the capsule.

In another aspect hereof, in the system in accordance with any other aspect hereof, the flexible region of the capsule is positioned approximately 43.5 mm from a proximal end of the capsule.

In another aspect hereof, in the system in accordance with any other aspect hereof, the catheter portion further incudes a middle shaft extending from the control handle portion and positioned within a first lumen formed by the outer shaft, and an inner shaft extending from the control handle portion and positioned within a second lumen formed by the middle shaft.

In another aspect hereof, in the system in accordance with any other aspect hereof, the distal portion includes a spindle coupled to the middle shaft, and a tip coupled to the inner shaft, wherein the tip is positioned distally from the spindle to define a space for receiving the implantable medical device between the spindle and the tip.

In another aspect hereof, in the system in accordance with any other aspect hereof, the tip is frustoconically shaped and tapers linearly from proximal end of the tip to a distal end of the tip.

In another aspect hereof, the system in accordance with any other aspect hereof further includes an introducer slidably positioned over the outer shaft, wherein the introducer includes an inline sheath, a hub coupled to proximal end of the inline sheath, and a stop cock coupled to the hub, wherein the stop cock is configured as a three way stop cock.

In another aspect hereof, a system for delivering an implantable medical device to an implant location includes a control handle portion, a catheter portion coupled to the control handle portion at a proximal end of the catheter portion, and a distal portion configured to receive the implantable medical device. The catheter portion includes an outer shaft, a capsule coupled to a distal end of the outer shaft, and an inner shaft. The capsule is actuatable to move axially from the distal portion to expose the implantable medical device. The capsule includes a ribbed member, a jacket laminated to a portion the ribbed member, and a flexibility region, the flexibility region having a stiffness that is less than other regions of the capsule due to delamination of the jacket from ribbed member in the flexibility region.

In another aspect hereof, in the system in accordance with any other aspect hereof, the flexibility region of the capsule is positioned approximately 43.5 mm from a proximal end of the capsule.

In another aspect hereof, in the system in accordance with any other aspect hereof, the jacket is delaminated from the ribbed member in the flexibility region by bending the capsule.

In another aspect hereof, in the system in accordance with any other aspect hereof, the outer shaft further includes an inner braided layer extending axially along the outer shaft; an outer braided layer extending axially along the outer shaft; an axial spine positioned between the inner braided layer and the outer braided layer extending axially along the outer shaft. A proximal portion of the outer shaft includes a first jacket layer surrounding the inner braided layer, the outer braided layer and the axial spine. A distal portion of the outer shaft includes a second jacket layer surrounding the inner braided layer, the outer braided layer and the axial spine. A middle portion of the outer shaft positioned between the proximal portion and the distal portion includes a third jacket layer surrounding the inner braided layer, the outer braided layer and the axial spine surrounding the inner braided layer, the outer braided layer and the axial spine.

In another aspect hereof, in the system in accordance with any other aspect hereof the first jacket layer, the second jacket layer, the third jacket layer comprise different materials.

In another aspect hereof, in the system in accordance with any other aspect hereof the axial spine comprises a single wire axially extending the length of the outer shaft.

In another aspect hereof, in the system in accordance with any other aspect hereof the catheter portion further includes a stability shaft coupled to the control handle portion and surrounding the outer shaft, wherein the stability shaft extends from the control handle portion to a distal position on the outer shaft.

In another aspect hereof, in the system in accordance with any other aspect hereof the distal position is approximately 36 inches from the control handle portion.

In another aspect hereof, a method of manufacturing a capsule for a delivery system includes forming the capsule comprising an inner liner, a ribbed member, and an outer jacket, wherein the outer jacket is laminated to the ribbed member, and bending the capsule at a predetermined position, wherein the bending delaminates the outer jacket from ribbed member located in a region around the predetermined position.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

The foregoing and other features and advantages of the present disclosure will be apparent from the following description of embodiments hereof as illustrated in the accompanying drawings. The accompanying drawings, which are incorporated herein and form a part of the specification, further serve to explain the principles of the present disclosure and to enable a person skilled in the pertinent art to make and use the embodiments of the present disclosure. The drawings are not to scale.

FIGS. 1A-1D depict illustrations of a delivery system for implantable medical devices, according to an embodiment hereof.

FIGS. 2A-2B depict illustrations of different enlarged views of the distal end and delivery catheter of the delivery system of FIGS. 1A-1D, according to an embodiment hereof.

FIGS. 3A and 3B depict illustrations of enlarged views of the tip and the inner shaft of the delivery system of FIGS. 1A-1D, according to an embodiment hereof.

FIGS. 4A, 4B, 4C, 5A, and 5B depict illustrations of several views of the capsule 112, according to an embodiment hereof.

FIGS. 6A-6E depict illustrations of several views of the outer shaft of the delivery system of FIGS. 1A-1D, according to an embodiment hereof.

FIGS. 7A-7F depict illustrations of several views of the stability shaft of the delivery system of FIGS. 1A-1D, according to an embodiment hereof.

FIGS. 8A-8C depict illustration of several views of the introducer of the delivery system of FIGS. 1A-1D, according to an embodiment hereof.

FIG. 9 depicts an illustration of a side view of the control handle portion 106 of the delivery system of FIGS. 1A-1D, according to an embodiment hereof.

FIGS. 10A-10C depict illustrations of a prosthetic heart valve that may be used with the delivery system of FIGS. 1A-1D, according to an embodiment hereof.

### DETAILED DESCRIPTION

Specific embodiments of the present disclosure are now described with reference to the figures. The following detailed description describes examples of embodiments and is not intended to limit the present technology or the application and uses of the present technology. Although the description of embodiments hereof is in the context of a delivery system, the present technology may also be used in other devices. Furthermore, there is no intention to be bound by any expressed or implied theory presented in the preceding technical field, background, brief summary or the following detailed description.

The terms “distal” and “proximal”, when used in the following description to refer to a delivery system or catheter are with respect to a position or direction relative to the treating clinician. Thus, “distal” and “distally” refer to positions distant from, or in a direction away from the treating clinician, and the terms “proximal” and “proximally” refer to positions near, or in a direction toward the clinician.

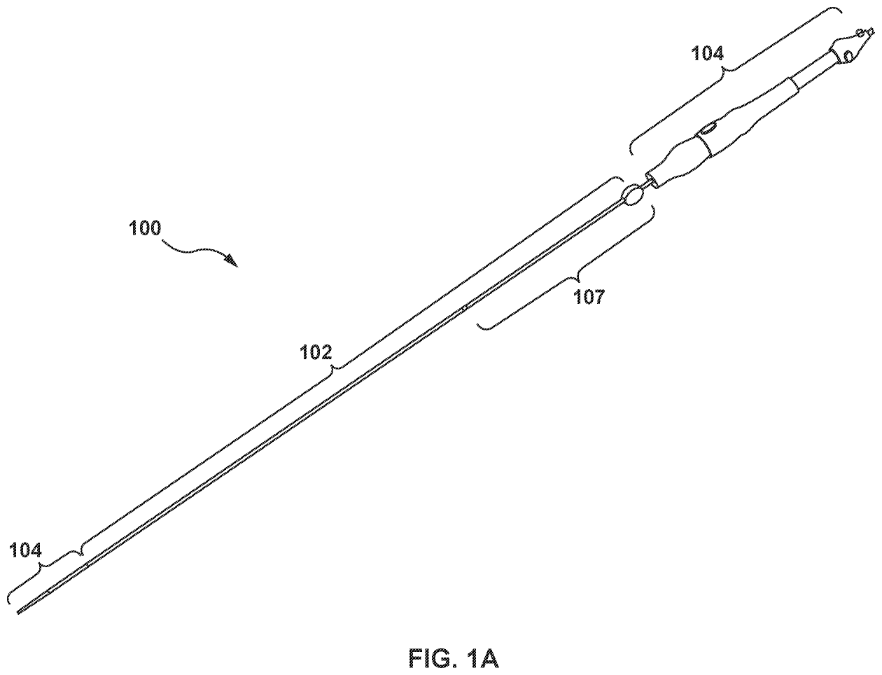

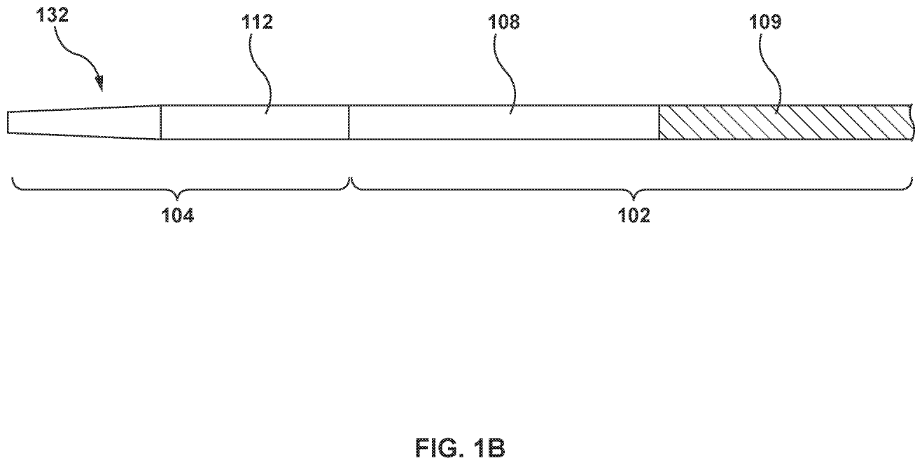

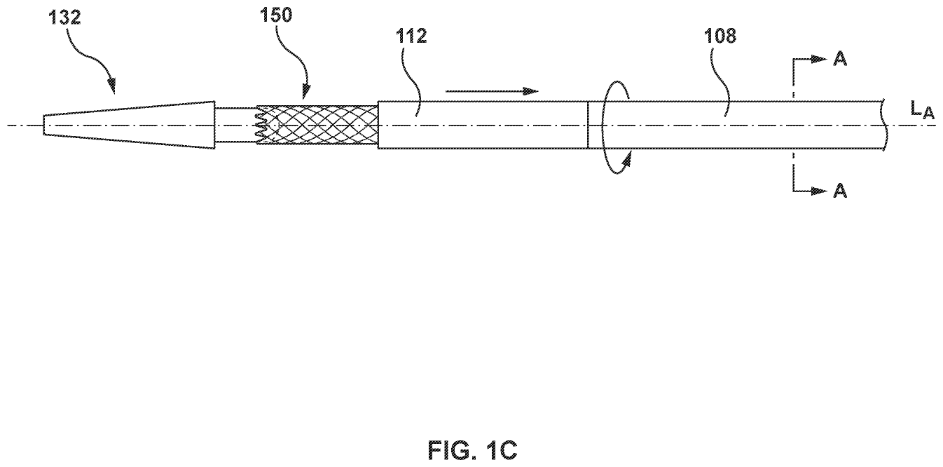

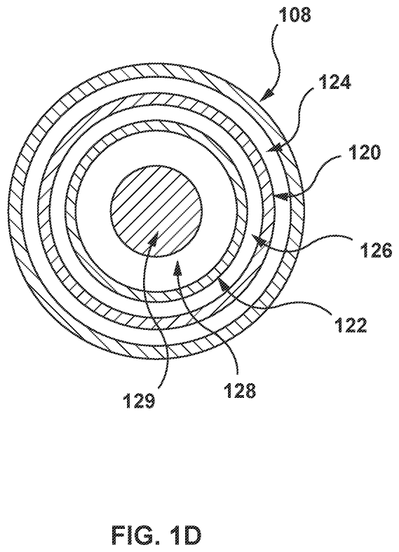

FIGS. 1A-1D illustrate an example of a delivery system 100 in accordance with an embodiment hereof. One skilled in the art will realize that FIGS. 1A-1D illustrate one example of a delivery system and that existing components illustrated in FIGS. 1A-1D may be removed and/or additional components may be added to the delivery system 100.

As shown in FIG. 1A, the delivery system 100 generally comprises a catheter portion 102, a distal portion 104, and a proximal control handle portion 106 by which the distal portion 104 is effectively controlled. The delivery system 100 also includes an introducer 107 that is configured to slide over portions of the catheter portion 102. The catheter portion 102 is preferably of a length and size so as to permit a controlled delivery of the distal portion 104 to a desired implantation location, for example, a patient's heart. The distal portion 104 provides the means by which an implantable medical device, e.g., a prosthetic heart valve, can be mounted for delivery to the implantation location and further provides for or allows the expansion of the implantable medical device for effective deployment thereof. The introducer 107 operates to provide an access lumen for introduction of the delivery catheter 102 and the distal end 104 including the implantable medical device to into a patient's body. The control handle portion 106 preferably controls movements as translated to the distal portion 104 by way of the elongate structure of the catheter portion 102. Controlled functionality from the control handle portion 106 is preferably provided in order to permit expansion and deployment of the implantable medical device at a desired location, such as a heart valve annulus, and to provide for ease in the delivery and withdrawal of the delivery system through a patient's vasculature.

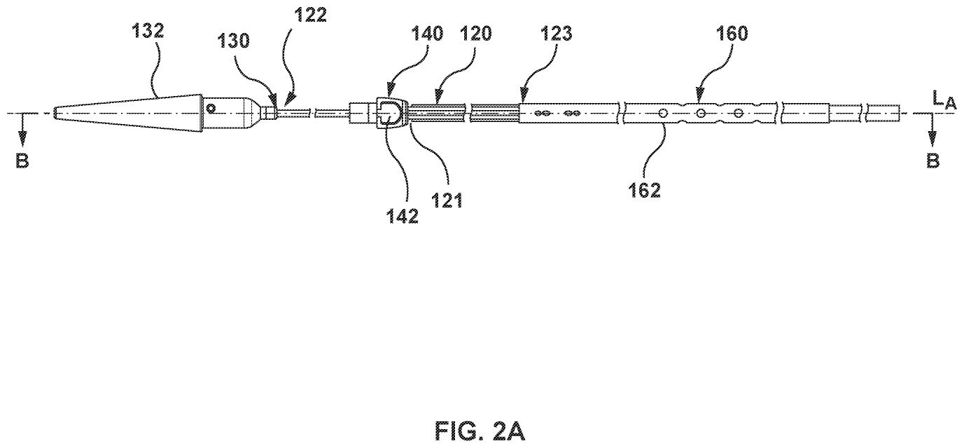

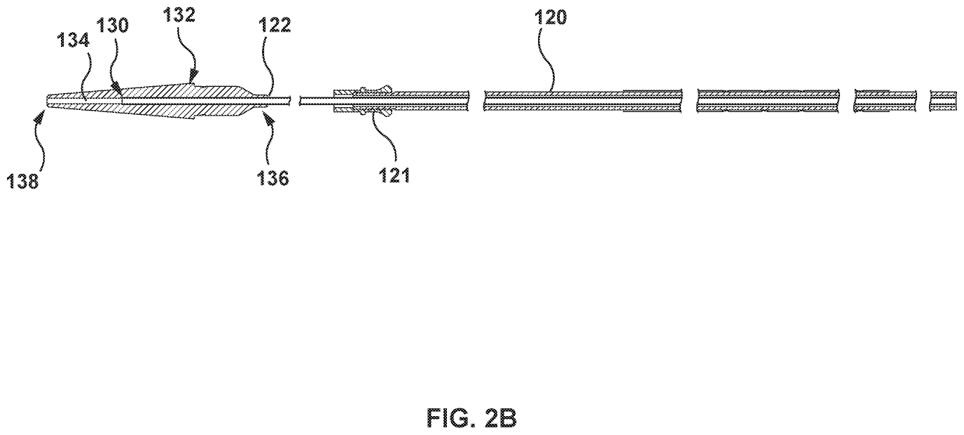

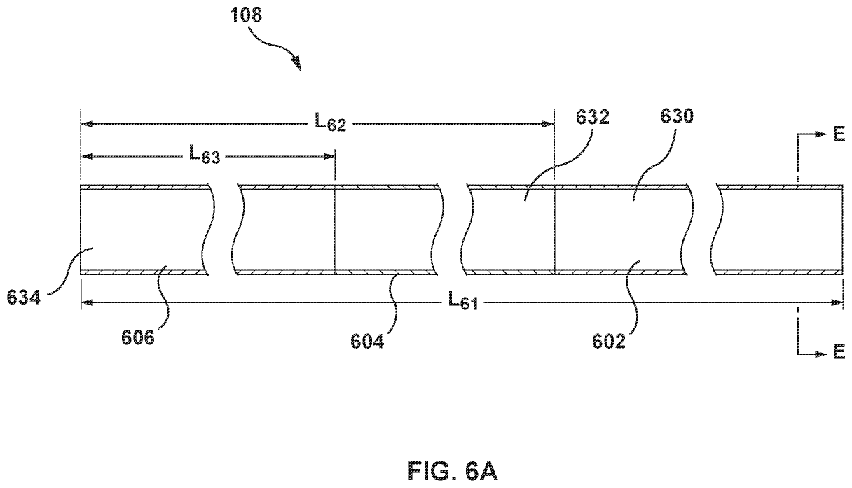

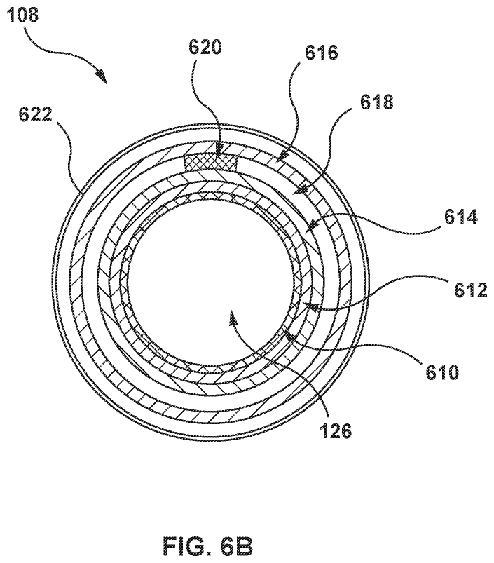

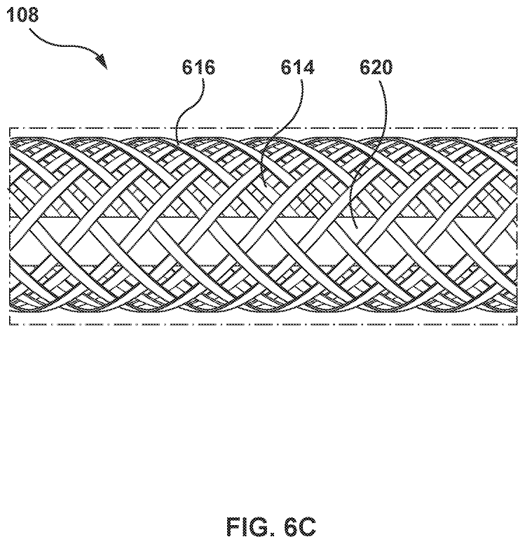

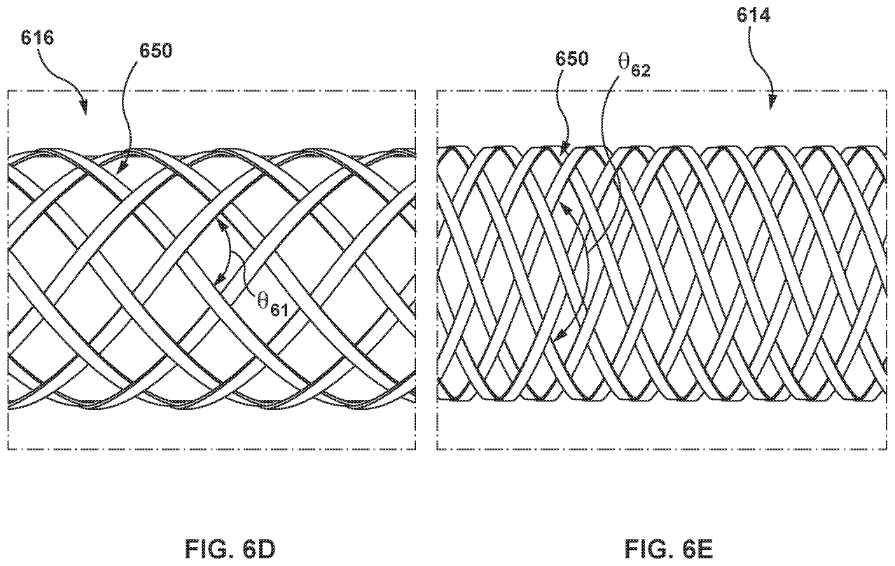

As illustrated in FIG. 1B, which is an enlarged view of the catheter portion 102 and distal portion 104 with the introducer 107 being removed, the catheter portion 102 of the delivery system 100 also preferably comprises an outer shaft 108 that is also operatively connected with the control handle portion 106 and that surrounds one or more inner shafts, e.g., a middle shaft 120 and an inner shaft 122 as discussed below in further detail FIGS. 2A-2B. In embodiments, the outer shaft 108 comprises one or more lubricous inner layers (such as high density polyethylene HDPE or Polytetrafluoroethylene PTFE), one or more braided stainless steel middle layers, an axial spine, and one or more flexible plastic outer layers, such as Pebax 7233, Pebax 6333, Nylon 12, Vestamid ML24, as described below in further detail with reference to FIGS. 6A-6C. The outer shaft 108 extends from the control handle portion 106 and facilitates the advancement of the delivery system 100 along a guide wire and through a patient's vasculature by improving the pushability of the delivery system 100 and by improving flexibility by including only a single axial spine.

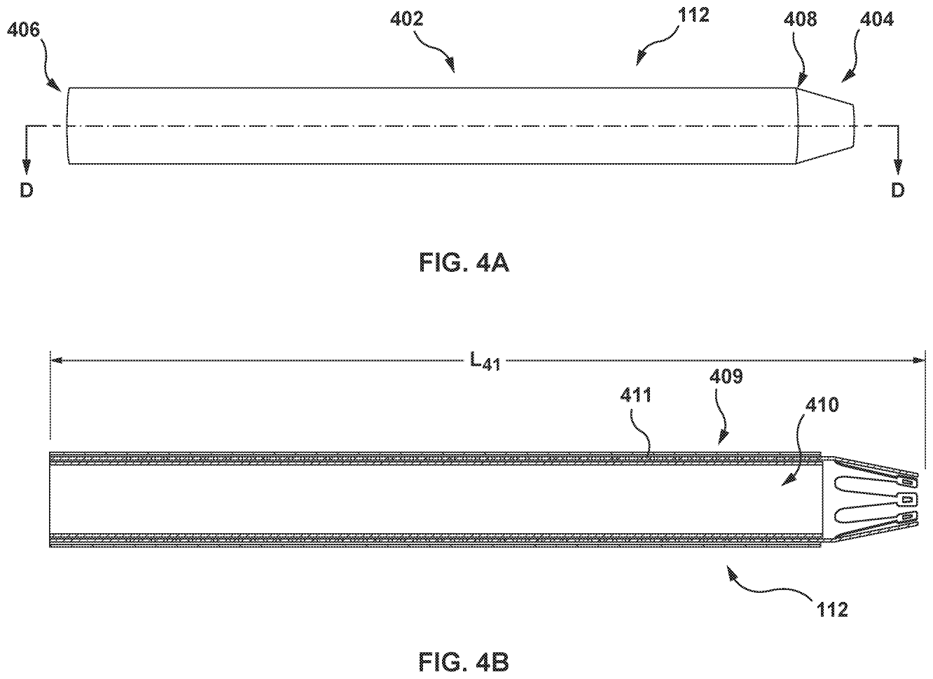

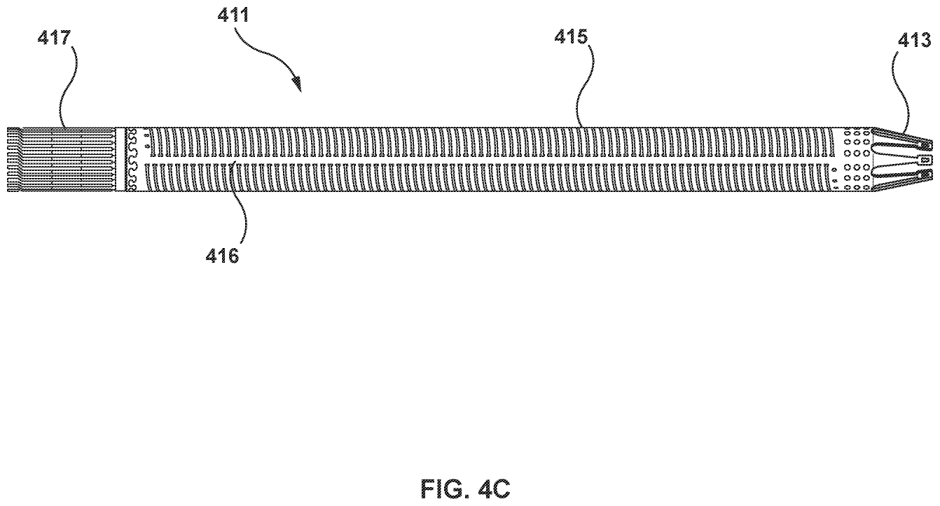

The outer shaft 108 is operatively coupled, at a proximal end, with the control handle portion 106 so as to be movable by operation of the handle control portion and that is connected with a sheath or capsule 112, as described below in further detail with reference to FIGS. 4A and 4B. In some embodiments, the capsule 112 can be a separate component that is coupled to the outer shaft 108. In some embodiments, the capsule 112 can be formed as an integrated extension of the outer shaft 108. The capsule 112 is configured to retain the implantable medical device, e.g., prosthetic heart valve, in a radially collapsed configuration for delivery to the desired implantation location as will be described in more detail below. That is, telescopic movement of the outer shaft 108 by operation of the control handle portion 106 results in the longitudinal translational movement of the capsule 112 proximally away from the distal portion 104, thereby exposing an implantable medical device, e.g., a self-expanding prosthetic heart valve 150, as illustrated in FIG. 1C. The control handle portion 106 is designed, among other things, for controlling the advancement and the withdrawal of the capsule 112.

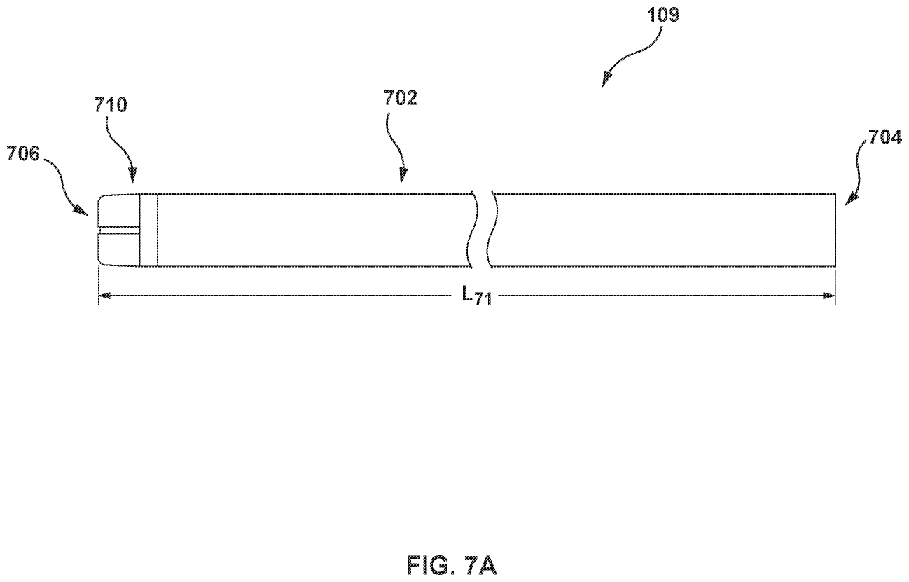

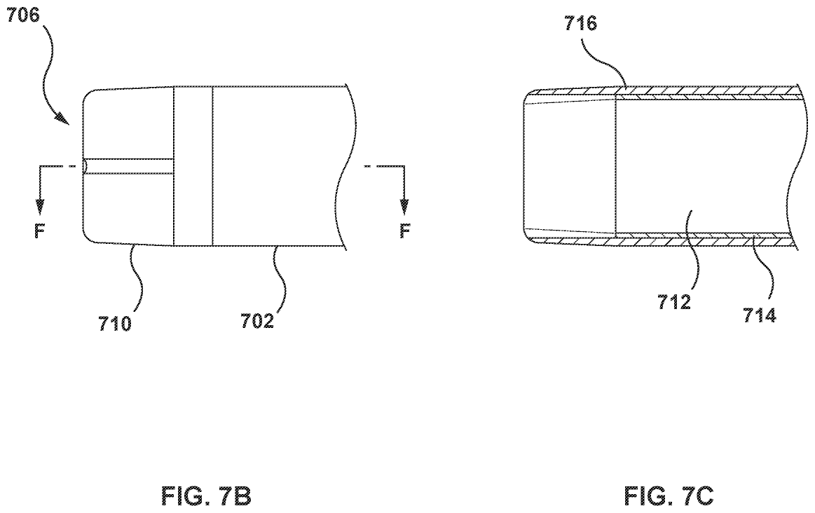

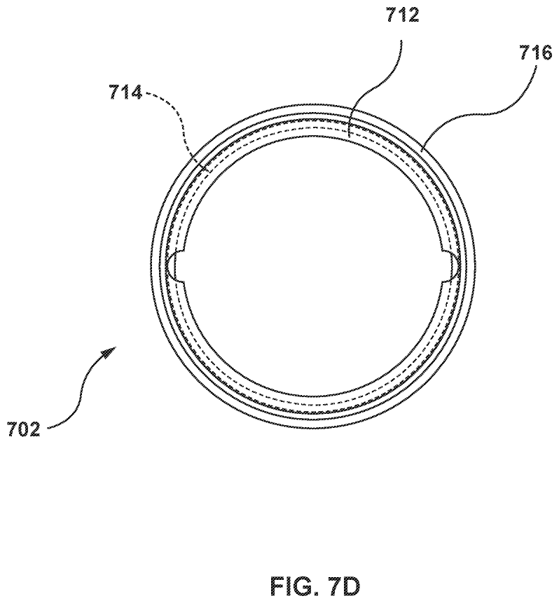

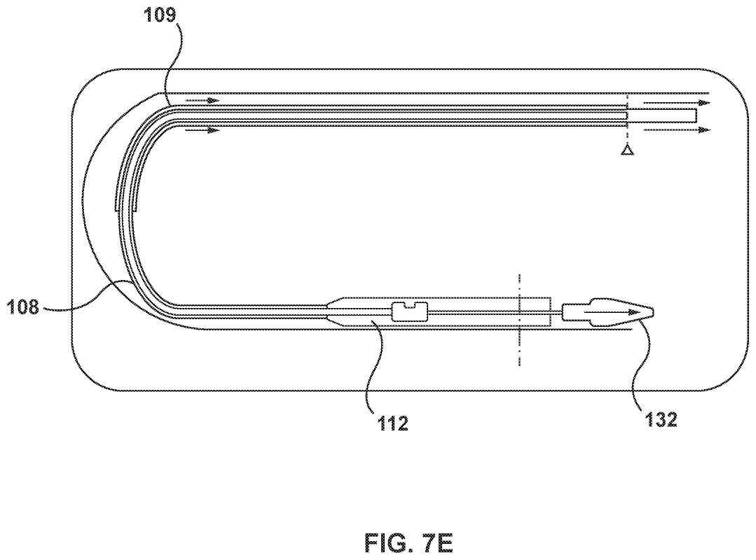

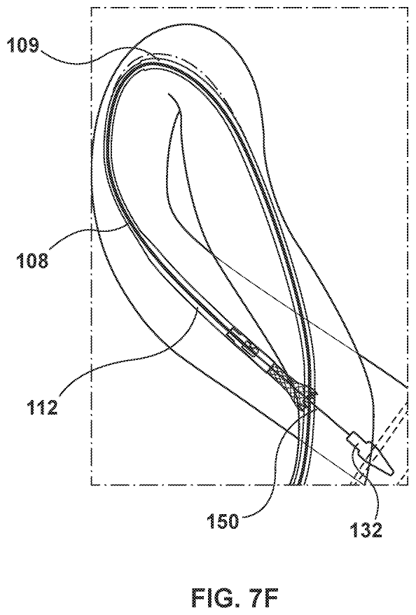

In embodiments, as shown in FIG. 1B, the catheter portion 102 of the delivery system 100 also includes a stability shaft 109. The stability shaft 109 is operatively coupled to a distal end of the control handle portion 106 and extends over a portion of the length of the outer shaft 108. In embodiments, the stability shaft 109 comprises a lubricous inner layer (such as high density polyethylene HDPE or Polytetrafluoroethylene PTFE), braided stainless steel middle layer with a flexible plastic outer layer, such as comprised of Vestamid Care ML24, Green PMS 368, Pebax 7233, or Nylon 12. The stability shaft 109 extends to a desired length of the catheter portion 102 of the delivery system 102 from the control handle portion 106, as described below in further detail with reference to FIGS. 7A-7E. The stability shaft 109 facilitates the advancement of the delivery system along a guide wire and through a patient's vasculature by improving the pushability of the delivery system 100. Also, the stability shaft 109 may add some stiffness to the proximal end of the catheter portion 102 which translates into a more supportive structure for the catheter portion 102. This stiffness of stability shaft 109 will minimize movement of the catheter portion 102 within the anatomy during the deployment of an implantable medical device as described below. For example, the stability shaft 109 as described herein aids the user in making a more accurate deployment of the self-expanding prosthetic heart valve 150 by limiting movement of the catheter portion 102 within the anatomy during deployment.

FIGS. 2A-2B illustrate different enlarged views of the distal end 104 and delivery catheter 102 in which the outer shaft 108, the capsule 112, and the stability shaft 109 are removed. As illustrated, in addition to the outer shaft 108 being operatively coupled to the control handle portion 106, the delivery device 100 further includes the middle shaft 120 that is slidingly disposed within the outer shaft 108 and is operatively coupled to the control handle portion 106. As used herein, “slidably” denotes back and forth (proximal and distal) movement in a longitudinal direction along or generally parallel to a central longitudinal axis LA of the delivery system 100. An inner shaft 122 is disposed within the middle shaft 120. As with the outer shaft 108, the middle shaft 120 and the inner shaft 122 each distally extend from within the control handle portion 106.

As illustrated in FIG. 1D, which is a cross-sectional view of the delivery catheter 102 taken along line A-A of FIG. 1C, the outer shaft 102 defines a lumen 124 and is slidingly and concentrically disposed over the middle shaft 120. The middle shaft 122 defines a lumen 126 and is concentrically disposed over the inner shaft 122. The inner shaft 122 defines a lumen 128 such that the delivery system 100 may be slidingly disposed and tracked over a guidewire 129.

The inner shaft 122 has a proximal end (not shown) which terminates within the control handle portion 106 and a distal end 130, as illustrated in FIG. 2B, which is a cross-section view taken along line B-B of FIG. 2A. A tapered flexible nosecone or distal tip 132 may be coupled to the distal end 130 of the inner shaft 122. In some embodiments, the distal end 130 of the inner shaft 122 can be located within a channel 134 that extends from a proximal end 136 to a distal end 138 of the distal tip 132.

Returning to FIG. 2A, the middle shaft 120 has a proximal end (not shown) disposed within the control handle portion 106 and a distal end 121 disposed inside of the capsule 112 when the capsule 112 is disposed over the implantable medical device. The distal end 121 of the middle shaft 120 may include a spindle 140 to which an end of the implantable medical device is releasably coupled. In some embodiments, the distal end 121 of the middle shaft 120 and spindle 140 can include matching male and female threads to attach the spindle 140 to the distal end 121 of the middle shaft 120. The inner shaft 122 is coupled to the middle shaft 120 at the spindle 140 such that the inner shaft 122 and the middle shaft 120 are slidingly disposed within the capsule 112 as an assembly.

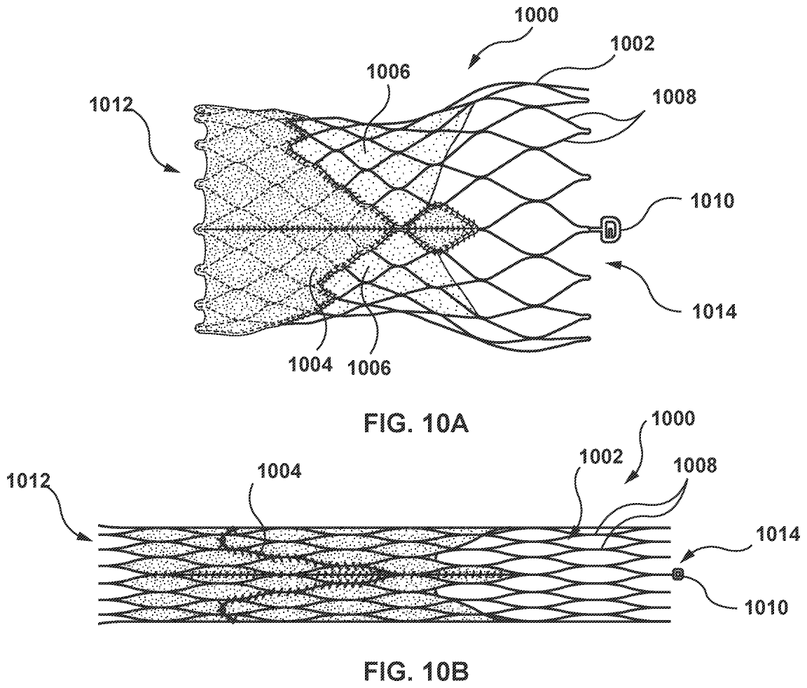

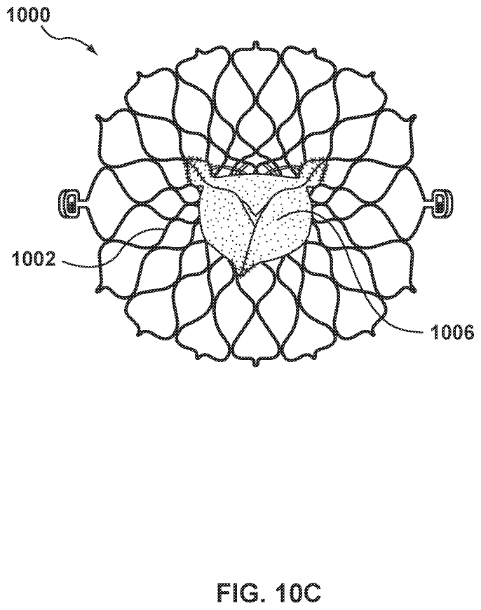

The spindle 140 is a tubular component having at least one recess 142, for example, two recesses 142, formed on an outer surface thereof that is configured to receive an attachment device (e.g., paddle) extending proximally from the implantable medical device, as descried below in further detail with reference to FIG. 10. The at least one recess 142 can be sized and shaped to closely correspond to the size and shape of attachment device of the implantable medical device, e.g., paddles of a prosthetic heart valve. The attachment device fits within or mates with the recess 142 of the spindle 140 such that the implantable medical device is releasably coupled to middle shaft 120. Although only one recess 142 is visible on FIG. 2A, it will be understood by one of ordinary skill in the art that the spindle 142 may include two or more recesses for receiving a mating paddle of the implantable medical device, such as for example first and second recesses at opposing circumferential locations on the spindle 140.

In embodiments, the inner shaft 122 is configured to receive the implantable medical device, e.g., a self-expanding prosthetic heart valve 150, on a distal portion thereof and the capsule 112 is configured to compressively retain the self-expanding prosthetic heart valve 150 on the distal portion of the inner shaft 122 during delivery. That is, the capsule 112 surrounds and constrains the self-expanding prosthetic heart valve 150 in a radially compressed or delivery configuration. As previously described, the distal end 121 of the middle shaft 120 includes the spindle 140 to which the self-expanding prosthetic heart valve is releasably coupled. During deployment of the self-expanding prosthetic heart valve in situ, the capsule 112 is proximally retracted with respect to the self-expanding prosthetic heart valve 150 via the control handle portion 106, thereby incrementally exposing the self-expanding prosthetic heart valve 150 until the self-expanding prosthetic heart valve 150 is fully exposed and thereby released from the delivery device 100, e.g., the spindle 140. That is, the middle shaft 120, the inner shaft 122 and the self-expanding prosthetic heart valve are held stationary while the outer shaft 108 and the capsule 112 are proximally retracted. When the capsule 112 is proximally retracted beyond the spindle 140, the attachment devices of the self-expanding prosthetic heart valve 150 are no longer held within the recesses 142 of the spindle 140 and the self-expanding prosthetic heart valve 150 is permitted to self-expand to its deployed configuration.

As further illustrated in FIG. 2A, in some embodiments, the middle shaft 120 can include a step 123 leading from a distal portion at a greater diameter than a proximal portion of the middle shaft 120. A flush tube 160 can be positioned over the smaller diameter proximal portion of the middle shaft 120 to the point of abutment with the step 123 at one end of the flush tube 160. The proximal end of the flush tube 160 can be coupled to a flush hub located in the control handle portion 106. The middle shaft 120 can also be coupled to the flush hub located in the control handle portion 106. For example, the flush tube 160 can have an inner diameter relative to the outer diameter of the middle shaft 120 so as to create an annular lumen between these respective surfaces so that flush fluid can be transported distally from the flush hub. With the connection of the flush hub of the control handle portion 106 to the flush tube 160, flush fluid can enter the lumen between the flush tube 160 and the middle shaft 120 to flow distally from that point of the delivery system 100.

For example, the flush tube 160 can include openings 162 that are preferably provided near the step 123 of the middle shaft 120. The openings 162 can allow for fluid flow from the lumen between the flush tube 160 and the middle shaft 120 by way of the openings 162. As such, the openings 162 can provide for fluid communication into the lumen 124 that is formed between the middle shaft 120 and the outer shaft 108. Accordingly, the flush fluid can communicate to the inside of the capsule 112. This fluid path allows for flushing of the entire length of the delivery system 100 through the implantable medical device for removing air from the system. With respect to each of the connections of the stability shaft 109, the outer shaft 108, the inner shaft 122, and the flush tube 160 to other components of the system, an adhesive bonding connections can be utilized for fixing the shaft or tube ends in place. One skilled in the art, however, will realize that other bonding or securement techniques and procedures could instead be utilized.

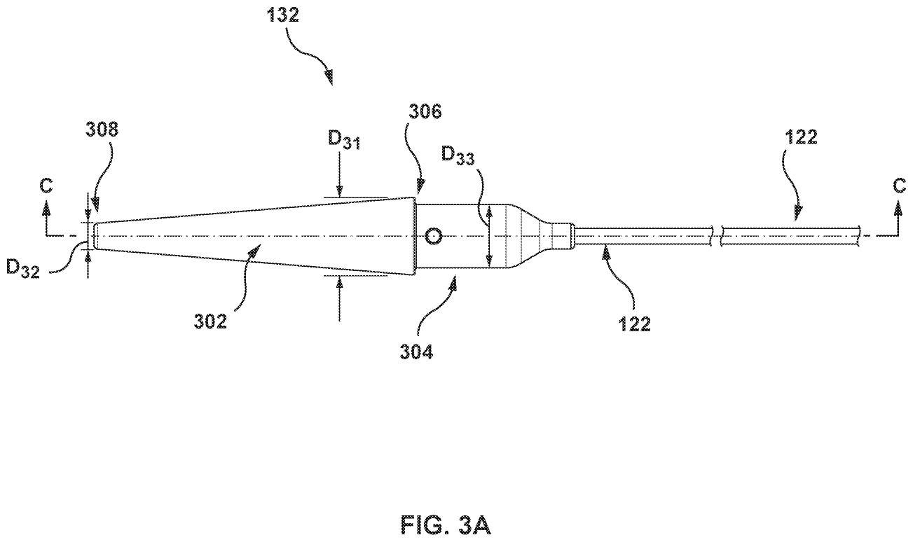

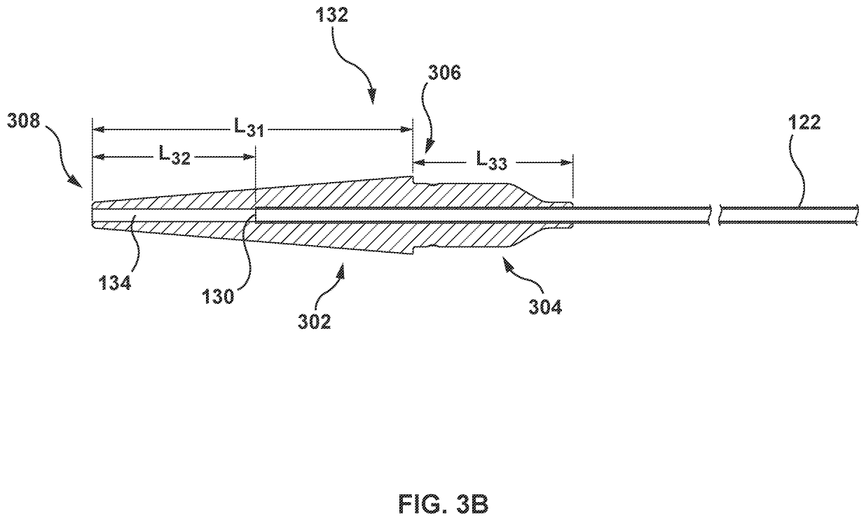

FIGS. 3A and 3B illustrate enlarged views of the tip 132 and the inner shaft 122. As shown in FIG. 3A, the tip 132 can include a distal portion 302 and a proximal portion 304. The distal portion 302 of the tip 132 can include a proximal end 306 that is coupled to the proximal portion 304 and a distal end 308 located at the distal end of the delivery system 100. In certain embodiments, the distal portion 302 and the proximal portion 304 are formed of a unitary piece, such as by molding. The proximal portion 304 of the tip 132 can be formed to a diameter that allows the capsule 112 to slide over the proximal portion 304 of the tip 132 and abut the proximal end 306 of the distal portion 302. The distal portion 302 of the tip 132 can have a frustoconical shape or tapered shape that decreases linearly in diameter from the proximal end 306 to the distal end 308 of the tip 132. The frustoconical shape of the distal portion 302 of the tip 132 improves ease of insertion of the distal end 104 of the delivery system 100 thereby improving deliverability. In an embodiment, the distal portion 302 can be formed of a soft polymeric material allowing for engagement with tissue of a vascular system during insertion, withdrawal, and maneuvering. In some embodiments, the distal portion 304 of the tip can be coated with a hydrophilic material. The tip 132 is shaped like an access dilator in that it is longer with a smaller angled taper. This provides smoother insertion into the access site and into the arteries (e.g., femoral/iliac arteries).

In embodiments, the distal portion 302 and the proximal portion 304 can be formed to various dimensions to accommodate different types and sizes of the implantable medical device. For example, in a first configuration, the proximal portion 304 can be formed to a diameter, D₃₃, of approximately 0.190 inch, and a length, L₃₃, of approximately 0.492 inch, as illustrated in FIG. 3B, which is a cross-sectional view taken along line C. In the first configuration, the distal portion 302 can be formed to a diameter, D₃₁, at the proximal end 306 of approximately 0.233 inch and a diameter, D₃₂, at the distal end 308 of approximately 0.07 inch. The distal portion 302 can also be formed to a length, L₃₁, from the proximal end 306 to the distal end 308 of approximately 0.984 inch, and a length, L₃₂, from the distal end 308 to the distal end 130 of the inner shaft 122 of approximately 0.0496 inch. In another example, in a second configuration, the proximal portion 304 can be formed to a diameter, D₃₃, of approximately 0.251 inch, and a length, L₃₃, of approximately 0.495 inch. In the second configuration, the distal portion 302 can be formed to a diameter, D₃₁, at the proximal end 306 of approximately 0.289 inch and a diameter, D₃₂, at the distal end 308 of approximately 0.07 inch. The distal portion 302 can also be formed to a length, L₃₁, from the proximal end 306 to the distal end 308 of approximately 1.063 inches, and a length, L₃₂, from the distal end 308 to the distal end 130 of the inner shaft of approximately 0.0498 inch. One skilled in the art will realize that any examples of dimensions described herein are approximate values and can vary by, for example, +/−5.0%, based on manufacturing tolerances, operating conditions, and/or other factors.

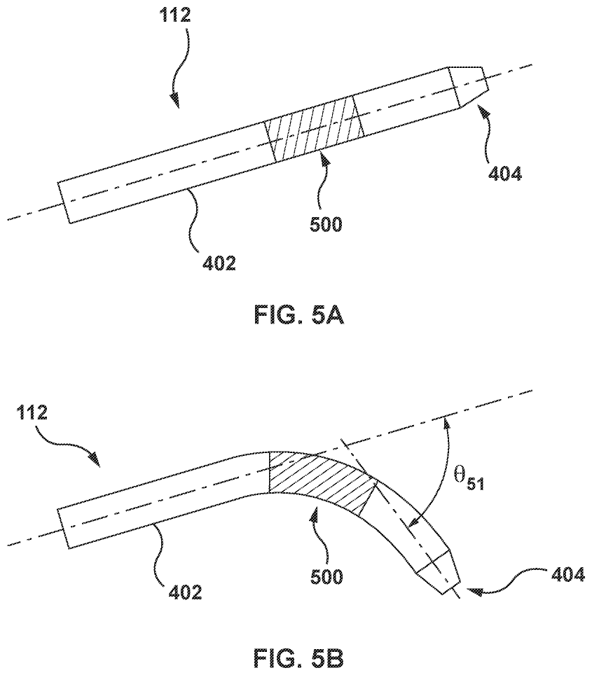

FIGS. 4A, 4B, 5A, and 5B illustrate several views of the capsule 112. As illustrated in FIG. 4A, which is a side view, the capsule 112 includes a body portion 402 having a proximal end 408 and a distal end 406. The capsule 112 also includes a tapered proximal portion 404 that is coupled to the proximal end 408 of the body portion 402. The tapered proximal portion 404 is also adapted to couple to the outer shaft 108. The tapered proximal portion 404 of the capsule 112 tapers from the larger diameter capsule 112 to the smaller diameter outer shaft 108. As illustrated in FIG. 4B, which is a cross-sectional view taken along line D-D of FIG. 4A, the body portion 402 is formed in a tubular shape that includes a ribbed member 411, a capsule jacket 409, a capsule liner 410. The ribbed member 411 is shown in more detail in FIG. 4C and includes a proximal tapered portion 413, a central portion 415, and a distal flared portion 417. A plurality of slots forms struts or ribs in the ribbed member 411. The central portion 415 of the ribbed member 411 includes two spines 416, although only one spine 416 is shown in FIG. 4C.

In embodiments, the capsule jacket 409 can be formed of a polymer material or a combination of polymer materials. For example, for some configurations, the capsule jacket 409 can be formed of a material composition comprising 66% Elasthane 80A, 20% Siloxane MB50-017, 10% Orevac, and 4% Foster Medibatch White. In embodiments, the ribbed member 411 can be formed of a rigid or semi-rigid materials such as a metals or metal alloys. For example, the ribbed member 411 can be formed of a nickel titanium alloy (e.g., Nitinol). The capsule liner 410 can be formed of a polymer material or a combination of polymer materials. For example, for some configurations, the capsule liner 410 can be formed of such as high density polyethylene HDPE or Polytetrafluoroethylene PTFE.

The capsule jacket 409 and the capsule liner 410 may be reflowed during manufacture, and openings in the ribbed member 411 allow reflow material (material of the capsule jacket 409 and capsule liner 410 in semi liquid form) to pass therethrough. As a result, the capsule jacket 409 and the capsule liner 410 fuse or join together during the reflow process in order to encapsulate the ribbed member 411.

In embodiments, the capsule 112 can be formed to various dimensions to accommodate different types and sizes of the implantable medical device. For example, in an embodiment, the capsule 112 can be formed to a length, L₄₁, that ranges between 3.3 inches and 3.8 inches. Likewise, in a second example, the capsule 112 can be formed to a length, L₄₁, that ranges between 4.25 inches and 4.75 inches.

In embodiments, as the distal portion 104 of the delivery system 100 is tracked to the implant location, the capsule 112 is required to undergo bending in order to track through the native anatomy. The capsule 112, however, can be formed of materials that resist bending. As such, as illustrated in FIG. 5A, the capsule 112 can undergo conditioning to create a flexibility region 500 in the capsule 112 prior to use in the delivery system 100. The flexibility region 500 has been conditioned to decrease the stiffness of the flexibility region 500 relative to other portions of the capsule 112. As illustrated in FIG. 5B, to condition the capsule 112, the capsule 112 can be bent at an angle θ51 relative to a central axis of the capsule 112. In some embodiments, the angle θ51 can range between 45 degrees and 60 degrees, preferably 53 degrees. As described above, because the capsule 112 is formed of layers of fused polymer layers, e.g., the capsule jacket 409 fused to the capsule liner 410 through the ribbed member 411, bending the capsule 112 in the flexibility region 500 causes the fused polymer materials to delaminate from the ribbed member 411 of the capsule 112, thereby reducing the stiffness of the capsule 112 in the flexibility region 500. In an embodiment, a linear length of the conditioned portion of the capsule is in the range of 16.5 mm. Further, the linear length may begin approximately 43.5 mm from a proximal end of the tapered proximal portion 404 of the capsule 112. The capsule 112 is bent in two directions, e.g. left and right side bend, to create the flexibility region 500. In an example, the flexural stiffness of the flexibility region 500 decreases in the range of 10% to 40%, or 15% to 35%, or 20% to 30% after bending as compared to the same region of the capsule prior to bending to create the flexibility region 500.

FIGS. 6A-6C illustrate several views of the outer shaft 108. As illustrated in FIG. 6A, the outer shaft 108 is formed as a tubular shape that extends from the control handle portion 106 to the capsule 112. The outer shaft 108 includes a proximal portion 602 coupled to an actuator of the control handle portion 106, a distal portion 606 positioned adjacent to the capsule 112, and a middle portion 604 positioned between the proximal portion 602 and the distal portion 606.

As illustrated in FIG. 6B, which is a cross-sectional view taken along line E-E, the outer shaft 108 is formed of a series of concentric layers that form a wall of the outer shaft 108, the inner surface of which defines the lumen 126. In embodiments, the outer shaft 108 includes one or more lubricous inner layers (such as high density polyethylene HDPE or Polytetrafluoroethylene PTFE) formed adjacent to the lumen 126 to allow ease of movement of the outer shaft 108 relative to the middle shaft 120. For example, the outer shaft 108 can include a first liner layer 610 and second liner layer 612. To provide structure to the outer shaft 108, the outer shaft includes an inner braided layer 614 and an outer braided layer 616, as illustrated in FIG. 6B. A middle liner layer 616 is formed between the inner braided layer 614 and the outer braided layer 616. Additionally, to provide support, a single axial spine 620 is positioned within the middle liner layer 616 between the inner braided layer 608 and the outer braided layer 610. One skilled in the art will realize that the outer shaft 108 can include one or more additionally liner layers positioned between the lubricous inner layer, the inner braided layer 614, the axial spine 620, and the outer braided layer 616.

As illustrated in FIG. 6B, the outer shaft 108 includes a jacket layer 622 formed as the outermost layer. As illustrated in FIG. 6A, the outer shaft 108 can also include a jacket layer 622 can include three sections: a proximal jacket layer 630 formed over the proximal portion 602, a middle jacket layer 632 formed over the middle portion 604, and a distal jacket layer 634 formed over the distal portion 606. In embodiments, the proximal jacket layer 630, the middle jacket layer 632, and the distal jacket layer 634 can be formed of different materials. For example, the proximal jacket layer 630 can be formed of Vestamid, the middle jacket layer 632 can be formed of Pebax 72D, and the distal jacket layer 634 can be formed of Pebax 63D. In this example, the use of the Pebax 63D for the distal jacket layer allows greater flexibility at the distal end of the outer shaft 108 and the use of Vestamid for the proximal jacket layer reduces compression in the outer shaft 108.

As illustrated in FIG. 6C, which is a side view with the liner layers removed, the inner braided layer 614, the outer braided layer 616, and the axial spine 620 extend the length of the outer shaft 108 axially from the control handle portion 106 to the capsule 112. In embodiments, the single axial spine 620 can be formed of materials that provide structural support to the outer shaft 108. For example, the single axial spine 620 can be formed of a metal or metal alloy such as stainless steel. The single axial spine 620 improves trackability/deliverability as compared to having multiple spines, such as two spines spaced 180° apart from each other.

FIG. 6D illustrates a side view of the outer braided layer 616, and FIG. 6E illustrates a side view of the inner braided layer 914. As illustrated, the outer braided layer 616 and the inner braided layer 614 are constructed as interlocking strips or wires 650 of material that form a mesh of the material, wherein a first series of the strips or wires 650 are oriented in a first direction and a second series of the strips or wires 650 are oriented in a second direction. For example, the outer braided layer 616 and the inner braided layer 614 can be constructed of interlocking strips 650 of a metal or metal alloy, such as stainless steel. In embodiments, to improve the stability of the outer shaft 108, the inner braided layer 614 can be formed having a higher density of the strips or wires 650 (higher or larger) braid angle) relative to the outer braided layer 616. For example, the inner braided layer 614 can be formed having a braid density of approximately 40 picks per inch (PPI) with a braid angle θ₆₂ that ranges from approximately 130 degrees to 140 degrees. The outer braided layer 616 can be formed having a braid density of approximately 20 PPI with a braid angle θ₆₁ that ranges from approximately 70 degrees to 80 degrees. The higher braid angle of the inner braided layer 614 maintains shaft flexibility. The lower braid angle of the outer braided layer 616 increases shaft rigidity and a resists polymer compressions in an axial direction.

In embodiments, the outer shaft 108 can be formed to various dimensions to accommodate different types and sizes of the implantable medical device. For example, as illustrated in FIG. 6A, in some configurations, the outer shaft 108 can be formed to a length, L₆₁, of 46.81 inches, the combination of the distal portion 606 and the middle portion 604 can be formed to a length, L₆₂, of 9.8 inches, and the distal portion 608 can be formed to a length, L₆₂, of 3.9 inches.

FIGS. 7A-7D illustrate several views of the stability shaft 109. As illustrated in FIG. 7A, the stability shaft 109 includes a body portion 702 having a proximal end 704 and a distal end 706. As illustrated in FIG. 7B, the stability shaft 109 can include a shaft tip 710 formed at the distal end 706. As illustrated in FIG. 7C, which is a cross-sectional view taken along line F-F, the body portion 702 of the stability shaft 109 includes a braided layer 714 having a liner layer 712 formed on the interior surface of the braided layer 714 and a jacket layer 716 formed on the outer surfaces of the braided layer 714. The liner layer 712 can be formed of lubricous material such as PTFE. The braided layer 714 can be formed of a metal or metal alloy such as stainless steel. The jacket layer 716 can be formed of Vestamid.

In embodiments, the stability shaft 109 can be formed to various dimensions to accommodate different types and sizes of the implantable medical device. In embodiments, the stability shaft 109 is formed to a length to improve stability of the catheter portion 102 for different anatomies. For a prosthetic heart valve deployment, ventricular movement during the initial stages of deployment may be common, which requires correction by a user through manipulation of the delivery device 100. For example, for delivery and deployment of a prosthetic aortic heart valve, the delivery catheter 102 of the delivery system 100 undergo significant bending as it travels though the aorta, as illustrated in FIG. 7E, which is a simplified representation of the path of the delivery catheter. As illustrated in FIG. 7E, friction of the capsule 112 along the anatomy may prevent movement of the capsule 112. When the outer shaft 108 is retracted, the catheter portion 102 moves towards the inner curvature of the aorta. In other words, instead of retracting, the outer shaft 108 moves inward and the capsule 122 remains stationary. Because the middle shaft 120 can move relative to the outer shaft 108, the middle shaft 120 pushes forward to compensate for the new path, thereby causing the tip 132 to potentially enter the heart.

As illustrated in FIG. 7F, the increased stiffness and length of the stability shaft 109 counteracts this movement. That is, the increased stiffness and length of the stability shaft 109 resists the movement toward the inner curvature. In embodiments, as illustrated in FIG. 7A, in some configurations, the stability shaft 109 can be formed to a length, L₇₁, of 36.5 inches. Likewise, the braided layer 714 can be formed having braid dimensions of 0.0003 in by 0.005 in. In an embodiment, the stability shaft 109 is about 80% the length of the outer shaft 108 distal of the handle portion 106. The stability shaft 109 provides increased stiffness within the geometrical constraints of fitting between the outer shaft 108 disposed within the stability shaft 109 and the in-line sheath 802, described below. Testing shows that the increased stiffness and length of the stability member 109 results in about a 70% decrease in longitudinal vascular movement of the inflow end of the transcatheter heart valve prosthesis for delivery system for a 29 mm nominal valve and about a 40% decrease in longitudinal vascular movement of the inflow end of the transcatheter heart valve prosthesis for a delivery system for a 34 mm nominal valve, thereby enabling a more accurate placement of the transcatheter heart prosthesis.

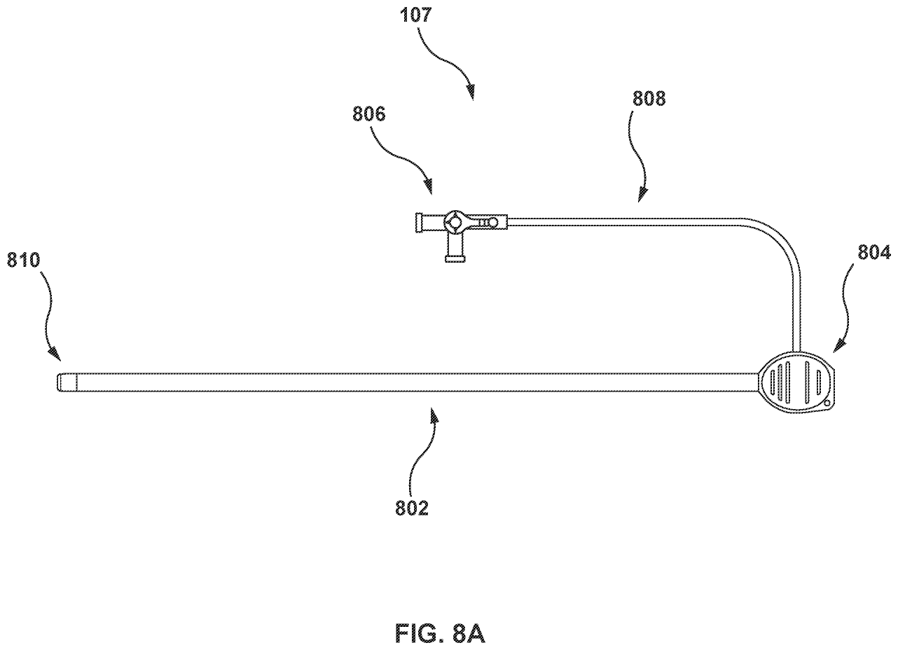

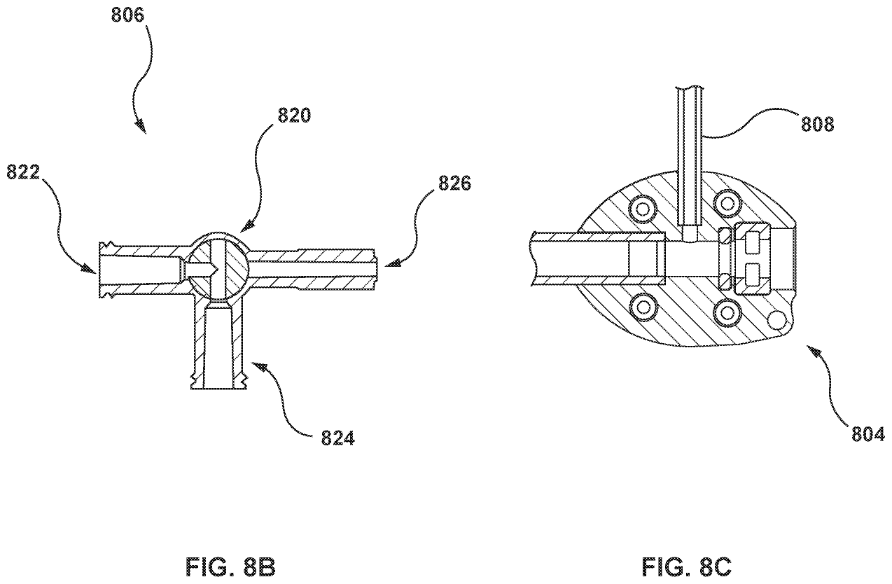

FIGS. 8A-8C illustrate of several views of the introducer 107. As illustrated in FIG. 8A, the introducer 107 includes an inline sheath 802, a hub 804, and a tip ring 810. The introducer 107 can also include a stop cock 806 coupled to the hub 804 by tubing 808. As further illustrated in FIG. 8C, which is a cross-sectional view, the stop cock 806 can include a valve 820 coupled between input ports 822 and 824 and an output port 826. The stop cock 806 can be actuated to place either input port 822 or input port 824 in fluid communication with the output port 826. The stop cock 806 can also be actuated to seal the output port 826 from the input ports 822 and 824. As such, fluid can be selective introduced to the hub 804 from either the input port 822 or the output port 824.

In embodiments, the inline sheath 802 can be slidably disposed about the stability shaft 109 and/or the outer shaft 108, which extend from control handle portion 106 to the distal portion 104. The inline sheath 802 can be made of any suitable material, for example, but not limited to, biocompatible plastic. In embodiments, the inline sheath 802 can include flexible and rigid portions. For example, in some embodiments, proximal and distal portions of the inline sheath 802 can be rigid while a middle portion of the inline sheath 802 between the proximal and distal portions can be flexible. In embodiments, the inline sheath 802 can be made of a coil reinforced shaft, for example, having a biocompatible polymer jacket, although this is not meant to be limiting. In some embodiments, the coil reinforcing element can be a steel, flat wire, but this is not meant to be limiting. The variability in flexibility described above can be achieved by varying the pitch of the coil. In certain embodiments, the inline sheath 802 can include a welded coil end to prevent flaring.

In embodiments, the inline sheath 802 can be formed of materials that reduce friction between the components, which can allow inline sheath 802 to slide easily along the stability shaft 109 and/or the outer shaft 108. As illustrated in FIG. 8C, which is a cross-sectional view, the inline sheath can include a liner on an inner surface thereof to reduce friction with the stability shaft 109 and/or the outer shaft 108. In embodiments, the tip ring 810 can be located at a distal end of inline sheath 802 to create an atraumatic transition with the stability shaft and the capsule.

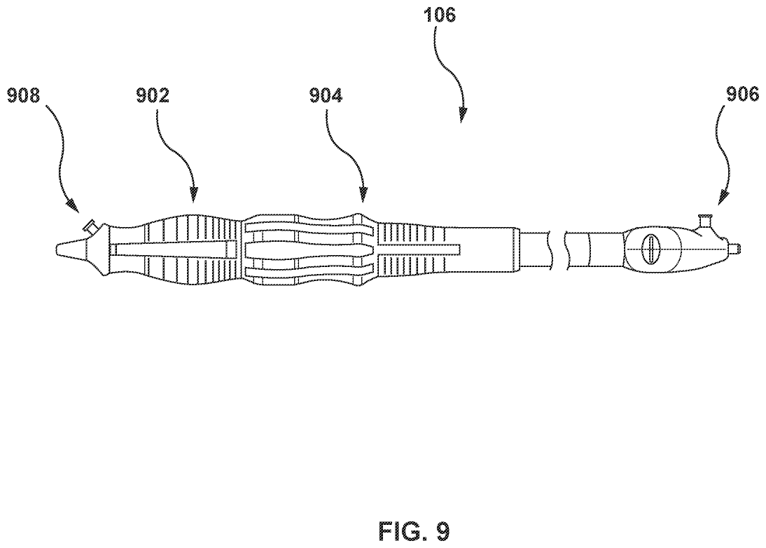

FIG. 9 illustrates a side view of the control handle portion 106. As illustrated, the control handle portion 106 includes a base 902 and an actuator 904. The actuator 904 can be utilized to advance and retract the outer shaft 108 and thereby advance and retract the capsule 112. For example, during deployment of the self-expanding prosthetic heart valve in situ, the actuator 904 can be rotated in order proximally retract the outer shaft 108 and the capsule 112 with respect to the self-expanding prosthetic heart valve 150. The actuator 904 can be incrementally rotated in order to incrementally expose the self-expanding prosthetic heart valve 150 until the self-expanding prosthetic heart valve 150 is fully exposed and thereby released from the delivery device 100, and prosthetic tabs 1010 have released from the spindle 140. Likewise, the outer shaft 108 and the capsule 112 can be advance by rotating the actuator 904 in an opposite direction.

Additionally, as shown, the control handle portion includes a proximal flush hub 906 and a distal flush hub 908. The proximal flush hub 906 can be in fluid communication with the flush tube 160, which has an inner diameter relative to the inner diameter of the middle shaft 120 so as to create an annular lumen between the middle shaft and the inner shaft so that flush fluid can be transported distally from the flush hub 908. With the connection of the proximal flush hub 906 of the control handle portion 106 to the flush tube 160, flush fluid can enter the lumen between the flush tube 160 and the middle shaft 120 to flow distally from that point of the delivery system 100.

In embodiments, the implantable medical devices useful with the present disclosure can be a prosthetic valve sold under the trade name CoreValve® available from Medtronic, Inc., Evolut™ Pro+ available from Medtronic, Inc., and the like. A non-limiting example of an implantable medical device useful with systems, devices and methods of the present disclosure is illustrated in FIGS. 10A-10C. In particular, FIG. 10A illustrates a side view of a prosthetic heart valve 1000 in a normal or expanded (uncompressed) arrangement. FIG. 10B illustrates the prosthetic heart valve 1000 in a compressed arrangement (e.g., when compressively retained within delivery system such as the distal portion 104 of the delivery system 100). The prosthetic heart valve 1000 includes a stent or frame 1002 and a valve structure 1004. The stent 1002 can assume any of the forms described above, and is generally constructed so as to be expandable from the compressed arrangement (FIG. 10B) to the uncompressed arrangement (FIG. 10A). In some embodiments, the stent 1002 is self-expanding. The valve structure 1004 is assembled to the stent 1002 and provides two or more (typically three) leaflets 1006, as illustrated in further detail below with reference to FIGS. 10C and 10D. The valve structure 1004 can be assembled to the stent 1002 in various manners, such as by sewing the valve structure 1004 to one or more of the wire segments or commissure posts defined by the stent 1002.

The prosthetic heart valve 1000 of FIGS. 10A and 10B can be configured to replace or repair an aortic valve. Alternatively, other shapes are also envisioned, adapted to the specific anatomy of the valve to be repaired (e.g., stented prosthetic heart valves in accordance with the present disclosure can be shaped and/or sized for replacing a native mitral, pulmonic, or tricuspid valve). With the example of FIGS. 10A and 10B, the valve structure 204 extends less than the entire length of the stent 1002, but in other embodiments can extend along an entirety, or a near entirety, of a length of the stent 1004. A wide variety of other constructions are also acceptable and within the scope of the present disclosure. For example, the stent 1002 can have a more cylindrical shape in the normal, expanded arrangement.

The stent 1002 includes support structures that comprise a number of struts or wire portions 1008 arranged relative to each other to provide a desired compressibility and strength to the valve structure 1004. The stent 1002 can also include one or more paddles 1010 that removably couple the prosthetic heart valve 1000 to a delivery system, e.g., the delivery system 100. While FIGS. 10A and 10B illustrate paddles 1010, one skilled in the art will realize that the paddles 1010 can be replaced with other components such as eyelets, loops, slots, or any other suitable coupling member. The paddles 1010 can include one or more radiopaque markers that aid in the positioning and orientation of the prosthetic heart valve 1000. The struts or wire portions 208 form a lumen having an inflow end 1012 and an outflow end 1014. Radiopaque markers may be includes, such as adjacent the inflow end 1012, to aid in depth alignment and/or rotational orientation. The struts or wire portions 1008 can be arranged such that the struts or wire portions 1008 are capable of transitioning from the compressed arrangement to the uncompressed arrangement. These wires are arranged in such a way that the stent 1002 allows for folding or compressing or crimping to the compressed arrangement in which the internal diameter is smaller than the internal diameter when in the uncompressed arrangement. In the compressed arrangement, such the stent 1002 with attached valve structure 1004 can be mounted onto a delivery system, such as the distal portion 104 the delivery system 100. The stent 1002 are configured so that they can be changed to an uncompressed arrangement when desired, such as by the relative movement of one or more sheaths relative to a length of the stent 1002.

In embodiments, the wires of the support structure of the stent 1002 in embodiments of the present disclosure can be formed from a shape memory material such as a nickel titanium alloy (e.g., Nitinol). With this material, the support structure is self-expandable from the compressed arrangement to the normal, expanded arrangement, such as by the application of heat, energy, and the like, or by the removal of external forces (e.g., compressive forces). This stent 1002 can also be compressed and re-expanded multiple times without significantly damaging the structure of the stent frame. In addition, the stent 1002 of such an embodiment may be laser-cut from a single piece of material or may be assembled from a number of different components or manufactured from a various other methods known in the art.

In embodiments, the stent 1002 can generally be tubular support structures having an internal area in which the leaflets 1006 can be secured. The leaflets 1006 can be formed from a variety of materials, such as autologous tissue, xenograph material, or synthetics as are known in the art. In some embodiments, the leaflets 1006 may be provided as a homogenous, biological valve structure, such as porcine, bovine, or equine valves. In some embodiments, the leaflets 1006 can be provided independent of one another and subsequently assembled to the support structure of the stent 1002. In some embodiments, the stent 1002 and the leaflets 1006 can be fabricated at the same time, such as may be accomplished using high-strength nano-manufactured NiTi films produced at Advanced Bioprosthetic Surfaces (ABPS), for example. The stent 1002 can be configured to accommodate at least two (typically three) of the leaflets 1006 but can incorporate more or fewer than three of the leaflets 1006.

FIG. 10C is an end view of FIG. 10A and illustrates an exemplary tricuspid valve having three leaflets 1006, although a bicuspid leaflet configuration may alternatively be used in embodiments hereof. More particularly, if the prosthetic heart valve 1000 is configured for placement within a native valve having three leaflets such as the aortic, tricuspid, or pulmonary valves, the transcatheter valve prosthesis 1000 includes three valve leaflets 1006. If the transcatheter valve prosthesis 1000 is configured for placement within a native valve having two leaflets such as the mitral valve, the prosthetic heart valve 1000 includes two valve leaflets 1006.

The leaflets 1006 may be made of pericardial material; however, the leaflets may instead be made of another material. Natural tissue for replacement valve leaflets may be obtained from, for example, heart valves, aortic roots, aortic walls, aortic leaflets, pericardial tissue, such as pericardial patches, bypass grafts, blood vessels, intestinal submucosal tissue, umbilical tissue and the like from humans or animals. Synthetic materials suitable for use as leaflets 1004 include DACRON® polyester commercially available from Invista North America S.A.R.L. of Wilmington, Del., other cloth materials, nylon blends, polymeric materials, and vacuum deposition nitinol fabricated materials. One polymeric material from which the leaflets can be made is an ultra-high molecular weight polyethylene material commercially available under the trade designation DYNEEMA from Royal DSM of the Netherlands. With certain leaflet materials, it may be desirable to coat one or both sides of the leaflet with a material that will prevent or minimize overgrowth. It is further desirable that the leaflet material is durable and not subject to stretching, deforming, or fatigue.

Delivery of the prosthetic heart valve 1000 may be accomplished via a percutaneous transfemoral approach or a transapical approach directly through the apex of the heart via a thoracotomy, or may be positioned within the desired area of the heart via different delivery methods known in the art for accessing heart valves. During delivery, if self-expanding, the prosthetic valve remains compressed until it reaches a target diseased native heart valve, at which time the prosthetic heart valve 1000 can be released from the delivery catheter and permitted to expand in situ via self-expansion. The delivery catheter is then removed and the prosthetic heart valve 1000 remains deployed within the native target heart valve.

In embodiments hereof, the stent 1002 is self-expanding to return to an expanded deployed state from a compressed or constricted delivery state and may be made from stainless steel, a pseudo-elastic metal such as a nickel titanium alloy or Nitinol, or a so-called super alloy, which may have a base metal of nickel, cobalt, chromium, or other metal. “Self-expanding” as used herein means that a structure/component has a mechanical memory to return to the expanded or deployed configuration. Mechanical memory may be imparted to the wire or tubular structure that forms stent 1002 by thermal treatment to achieve a spring temper in stainless steel, for example, or to set a shape memory in a susceptible metal alloy, such as nitinol, or a polymer, such as any of the polymers disclosed in U.S. Pat. Appl. Pub. No. 2004/0111111 to Lin, which is incorporated by reference herein in its entirety. Alternatively, the prosthetic heart valve 1000 may be made balloon-expandable as would be understood by one of ordinary skill in the art.

While the components of the delivery system 100 are described above with relative terms “first,” “second,” “proximal,” and “distal,” one skilled in the art will realize that the use of these terms is intended only to identify components of the delivery system 100 and do not define any preferred or ordinal arrangement of the components of delivery system 100.

As used herein, the term “approximately” when referring to dimensions means plus or minus 10% of the dimension.

It should be understood that various embodiments disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single device or component for purposes of clarity, it should be understood that the techniques of this disclosure may be performed by a combination of devices or components associated with, for example, a medical device.

What is claimed is:

1. A system for delivering an implantable medical device to an implant location, the system comprising:
a control handle portion;
a catheter portion coupled to the control handle portion at a proximal end of the catheter portion, the catheter portion comprising an outer shaft, wherein the outer shaft comprises:
an inner braided layer extending axially along the outer shaft,
an outer braided layer extending axially along the outer shaft, wherein the outer braided layer has a lower density of braids relative to the inner braided layer, and
an axial spine positioned between the inner braided layer and the outer braided layer extending axially along the outer shaft; and
a distal portion configured to receive the implantable medical device.

2. The system of claim 1, wherein the axial spine comprises a single wire axially extending the length of the outer shaft.

3. The system of claim 1, wherein the outer shaft further comprises:
a proximal portion including a first jacket layer surrounding the inner braided layer, the outer braided layer and the axial spine,
a distal portion positioned distal to the proximal portion and including a second jacket layer surrounding the inner braided layer, the outer braided layer and the axial spine, and
a middle portion positioned between the proximal portion and the distal portion and including a third jacket layer surrounding the inner braided layer, the outer braided layer and the axial spine surrounding the inner braided layer, the outer braided layer and the axial spine.

4. The system of claim 3, wherein the first jacket layer, the second jacket layer, the third jacket layer comprise different materials.

5. The system of claim 1, wherein the catheter portion further comprises:
a stability shaft coupled to the control handle portion and surrounding the outer shaft, wherein the stability shaft extends from the control handle portion to a distal position on the outer shaft.

6. The system of claim 5, wherein the distal position is approximately 36 inches from the control handle portion.

7. The system of claim 1, the system further comprising:
a capsule coupled to a distal end of the outer shaft, wherein the capsule is actuatable to move axially from the distal portion to expose the implantable medical device.

8. The system of claim 7, wherein the capsule comprises a flexible region, the flexible region having a stiffness that is less than other regions of the capsule.

9. The system of claim 8, wherein the flexible region of the capsule is positioned approximately 43.5 mm from a proximal end of the capsule.

10. The system of claim 1, wherein the catheter portion further comprises:
a middle shaft extending from the control handle portion and positioned within a first lumen formed by the outer shaft; and
an inner shaft extending from the control handle portion and positioned within a second lumen formed by the middle shaft.

11. The system of claim 9, wherein the distal portion comprises:
a spindle coupled to the middle shaft; and
a tip coupled to the inner shaft, wherein:
the tip is positioned distally from the spindle to define a space for receiving the implantable medical device.

12. The system of claim 11, wherein the tip is frustoconically shaped that tapers linearly from proximal end of the tip to a distal end of the tip.

13. The system of claim 1, the system further comprising:
an introducer slidably positioned over the outer shaft, wherein the introducer comprises:
an inline sheath,
a hub coupled to proximal end of the inline sheath, and
a stop cock coupled to the hub, wherein the stop cock is configured as a three way stop cock.

14. A system for delivering an implantable medical device to an implant location, the system comprising:
a control handle portion; and
a catheter portion coupled to the control handle portion at a proximal end of the catheter portion, the catheter portion comprising an outer shaft, a capsule coupled to a distal end of the outer shaft, and an inner shaft; and
a distal portion, the distal portion being configured to receive the implantable medical device between the inner shaft and the capsule,
wherein the capsule is actuatable to move axially from the distal portion to expose the implantable medical device and wherein the capsule is tubular shaped and comprises:
a ribbed member,
a jacket laminated to a portion the ribbed member, and
a flexibility region, the flexibility region having a stiffness that is less than other regions of the capsule due to delamination of the jacket from ribbed member in the flexibility region.

15. The system of claim 14, wherein the flexibility region of the capsule is positioned approximately 43.5 mm from a proximal end of the capsule.

16. The system of claim 14, wherein the jacket is delaminated from the ribbed member in the flexibility region by bending the capsule.

17. The system of claim 14, wherein the outer shaft further comprises:
an inner braided layer extending axially along the outer shaft;
an outer braided layer extending axially along the outer shaft; and
an axial spine positioned between the inner braided layer and the outer braided layer extending axially along the outer shaft;
wherein:
a proximal portion of the outer shaft further includes a first jacket layer surrounding the inner braided layer, the outer braided layer and the axial spine;
a distal portion of the outer shaft positioned proximal to the capsule includes a second jacket layer surrounding the inner braided layer, the outer braided layer and the axial spine; and
a middle portion of the outer shaft positioned between the proximal portion and the distal portion further includes a third jacket layer surrounding the inner braided layer, the outer braided layer and the axial spine surrounding the inner braided layer, the outer braided layer and the axial spine.

18. The system of claim 17, wherein the first jacket layer, the second jacket layer, the third jacket layer comprise different materials.

19. The system of claim 17, wherein the axial spine comprises a single wire axially extending the length of the outer shaft.

20. The system of claim 14, wherein the catheter portion further comprises:
a stability shaft coupled to the control handle portion and surrounding the outer shaft, wherein the stability shaft extends from the control handle portion to a distal position on the outer shaft.

21. The system of claim 20, wherein the distal position is approximately 36 inches from the control handle portion.

22. A method of manufacturing a capsule for a delivery system delivering an implantable medical device to an implant location, the system comprising:
forming the capsule comprising an inner liner, a ribbed member, and an outer jacket, wherein the outer jacket is laminated to the ribbed member; and
bending the capsule at a predetermined position, wherein the bending delaminates the outer jacket from ribbed member located in a region around the predetermined position.
